(19) United States
(12) Patent Application Publication
(10) Pub. No.: US 2015/0182659 A1
(43) Pub. Date: Jul. 2, 2015

(54) METHODS AND KITS FOR REVERSIBLE ADHESION OF IMPLANTS TO AN EYE SCLERA

(71) Applicant: Tel Hashomer Medical Research Infrastructure and Services Ltd., Ramat Gan (IL)

(72) Inventor: Ido Didi Fabian, Tel Aviv (IL)

(21) Appl. No.: 14/583,946

(22) Filed: Dec. 29, 2014

Related U.S. Application Data
(60) Provisional application No. 61/921,481, filed on Dec. 29, 2013.

(51) Int. Cl. A61L 24/10; A61N 5/10; A61F 9/00; A61K 38/48; A61F 9/007

(52) U.S. Cl. CPC A61L 24/106; A61K 38/482; A61F 9/007; A61F 9/0017; A61N 5/1027; C12Y 304/21031; A61N 2005/1024; A61F 2250/0067; A61F 2220/005

(57) ABSTRACT

Methods and kits are provided for reversibly affixing an implant to a sclera of an eye of a subject. The methods and kits are based on applying a composition comprising at least one fibrin-based tissue adhesive to at least one of said implant and said sclera, thereby affixing said implant to the sclera, and applying a composition comprising at least one plasminogen activator to at least one of said implant and said sclera following a desired time-period, thereby detaching said implant from said sclera, and removing said implant from said eye.

### FIELD OF THE INVENTION

The present invention relates, according to some embodiments, to methods and kits for affixing eye implants to the sclera of an eye via a fibrin-based tissue adhesive and for breaking down said fibrin-based tissue adhesive.

### BACKGROUND OF THE INVENTION

Brachytherapy, also known as sealed source radiotherapy, is a form of radiotherapy where a radiation source is placed inside or next to the area requiring treatment. Brachytherapy may be used in the eyes of patients suffering from eye neoplasms in order to prevent tumor growth. Brachytherapy may further be used for treatment of disorders such as choroidal neovascularization (CNV) in which arrest of neovascularization is desired.

Eye neoplasms may be primary neoplasms, which originate from within the eye, or metastatic neoplasms which spread to the eye from another organ. The two most common cancers that spread to the eye from another organ are breast cancer and lung cancer. The most common primary intraocular neoplasm in adults is uveal melanoma (UM) while retinoblastoma (RB) is the most prevalent in children. Treatments of small to medium UM tumors include enucleation of the eye (removal of at least part of the eye) and brachytherapy. Studies have shown that, in terms of survival and metastasis growth, the efficiency of treatment of a small to medium UM tumor using enucleation and brachytherapy is similar. Thus, brachytherapy is the most common treatment for UM. Brachytherapy is additionally used for treatment of retinoblastoma and in some cases of metastasis in the eye.

In order to use brachytherapy for treatment of UM, for example, radioactive seeds, such as Iodine-125 seeds are mounted onto a silicon mold which is inserted into a gold plaque, resulting in a radioactive plaque. The gold plaque, ranging 12-22 mm in diameter and 2.5-33 mm in height seals the radioactive seeds and prevent radiation from reaching non-treated areas. The radioactive plaque is then placed on the episclera of the eye, such that the radioactive seeds face the sclera. Sutures are used to affix the radioactive plaque to the sclera. The radioactive plaque is left in place for 3-7 days, after which the sutures need to be removed from the eye of the patient in order to extract the plaque.

Using sutures to affix the plaque to the sclera requires a highly skilled care giver which is able to insert a needle into the sclera without inducing spreading of the tumour cells or retinal detachment. Furthermore, affixing the plaque using sutures may result in movement of the plaque from the exact area to be treated, resulting in treatment failure (Almony A. et al., 2008, Arch. Opthalmol., 126(1):65-70). Moreover, the radiation may induce inflammation of the conjunctiva, which makes finding and removing the sutures difficult (Gunduz K et al., 2010, Clin. Ophthalmo., 24(4):159-161). Currently, affixing a plaque to the eye of a patient is mainly used for brachytherapy.

Fibrin glue, also known as fibrin sealant or fibrin adhesive, is a formulation which is used to create a fibrin clot which may be administered in order to glue tissues in various applications. Fibrin glue is formed by a reaction of fibrinogen, a blood coagulation protein, with thrombin, an enzyme which converts fibrinogen to fibrin. Fibrin glue is often used in applications in which irreversible sealing or gluing of tissues is desired, for example, repair of dura tears or achieving hemostasis after spleen and liver trauma.

Fibrin glue is also used in ophthalmologic applications requiring irreversible adhesion of tissue, such as conjunctival closure following pterygium and strabismus surgery, forniceal reconstruction surgery, amniotic membrane transplantation, lamellar corneal grafting, closure of corneal perforations and descematoceles, management of conjunctival wound leaks after trabeculectomy, lid surgery, adnexal surgery and as a hemostat to minimise bleeding. However, several studies indicated that adhesive strength using fibrin glue is lower than that achieved using sutures. For example, Shyam S. C et al. demonstrated that a conjunctival graft attached with sutures had higher adhesive strength compared with grafts glued with fibrin glue which was applied either spray or drop-wise (Shyam S. C. et al., 2012, Trans. Vis. Sci. Tech., 1(2):2).

Plasminogen activator is a protease which enables transformation of the inactive protein plasminogen to the active enzyme plasmin during the fibrinolysis cascade. Plasmin, in turn, is able to break down fibrin to fibrin degradation products, thus breaking down fibrin clots. Tissue-type plasminogen activator (tPA) is a serine protease found on blood endothelial cells, which functions to catalyse conversion of plasminogen to plasmin Since tPA has a fibrinolytic activity it is used in medical applications such as treatment of thrombotic or embolic stroke.

There is still a need, however, for a method for accurately, strongly and reversibly affixing an implant such as a plaque or a radioactive plaque to the sclera of a subject in need thereof.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

The present invention provides, according to some embodiments, methods for reversibly affixing an implant to a sclera of an eye using a highly resistant fibrin-based tissue adhesive. The present invention is based in part on the finding that using an exogenously-applied fibrin-based tissue adhesive to affix an implant to the sclera results in a surprisingly powerful adhesion of the implant, as exemplified herein below. The present invention is further based in part on the unexpected discovery that administration of a composition comprising at least one plasminogen activator to the site to which the exogenous fibrin-based tissue adhesive had been administered, results in breakdown of the fibrin-based tissue adhesive, enabling extraction of the implant.

In contrast to hitherto described clinical applications of fibrin glue for irreversible sealing or adhesion of tissues, the present invention discloses the use of fibrin glue for reversible gluing of an implant to the sclera of an eye, using a combination of fibrin glue and a plasminogen-activator.

According to some embodiments, the methods presented herein enable accurately placing an implant on a subject's sclera, strongly affixing the implant to the sclera for a desired time period and easily removing the implant at the end of treatment.

Advantageously, affixing an implant to the sclera of a subject according to methods presented herein is safe with regard to ocular surface tissue reaction and causes no elevation in intraocular pressure, as exemplified herein below. In addition, the implants show negligible horizontal movement and no tilting, thus facilitating treatment success.

When reducing the invention to practice the inventors found that, according to some embodiments, it is preferable to apply the fibrin-based tissue adhesive on top of the implant after the implant is placed on the sclera. As used herein, the “top surface” of the implant is the surface that is not facing the sclera. According to these embodiments the fibrin-based tissue adhesive is not applied to the space between the implant and sclera. According to these embodiments a layer of tissue adhesive is formed that preferably covers the top surface of the implant and a portion of the sclera in the immediate surroundings of the implant. The layer is preferably not formed beneath the implant, in the space between the implant and sclera. According to some embodiments, applying the fibrin-based tissue adhesive on top of the implant results in a layer of fibrin glue which covers the implant while binding both to the sclera and the conjunctiva. Thus, according to some embodiments, applying the fibrin-based tissue adhesive on top of the implant results in affixing the implant both to the sclera and the conjunctiva. According to some embodiments, applying the fibrin-based tissue adhesive on top of an implant enables firmly affixing implants which are inert to the fibrin-glue, such as a gold plaque, to the sclera and conjunctiva.

According to certain embodiments, the disclosed methods enable treating uveal melanoma and/or choroidal neovascularization (CNV) by placing a radioactive plaque at the site of uveal melanoma and/or CNV on a subject's sclera, affixing the plaque to the sclera using a composition comprising a fibrin-based adhesive and removing the plaque using a composition comprising plasminogen-activator at the end of treatment. Each possibility represents a separate embodiment of the present invention. According to some embodiments, treating CNV relates to a disease or condition comprising CNV in the eyes. According to some embodiments, a disease or condition comprising CNV in the eyes is age-related macular degeneration (AMD). According to some embodiments, a disease or condition comprising CNV in the eyes is selected from the group consisting of: AMD, ocular histoplasmosis syndrome, pathological myopia, Idiopathic polypoidal choroidal vasculopathy (IPCV), a Bruch's membrane defect, angioid streaks, vitelliform macular dystrophy, fundus flavimaculatus, optic nerve head drusen, multifocal choroiditis, serpignous choroiditis, toxoplasmosis, toxocariasis, rubella, Vogt-Koyanagi-Harada syndrome, Behcet syndrome, sympathetic opthalmia, choroidal nevus, choroidal hamangioma, metastatic choroidal tumor, hemartoma of the RPE, choroidal rupture, intense photocoagulation and a combination thereof. Each possibility represents a separate embodiment of the present invention.

According to other embodiments, the disclosed methods enable treating myopia by placing a plaque which comprises a pharmaceutical composition comprising riboflavin on a subject's sclera, affixing the plaque to the sclera using a composition comprising a fibrin-based adhesive and removing the plaque using a composition comprising plasminogen-activator at the end of treatment. According to some embodiments, the method of treating myopia according to the present invention further comprises irradiating the sclera using ultra violet radiation (herein after “UVA radiation”).

According to one aspect, the present invention provides a method for reversibly affixing an implant to a sclera of an eye, the method comprising: placing an implant on said sclera; and
applying a composition comprising at least one fibrin-based tissue adhesive to at least one of said implant and said sclera, thereby affixing said implant to the sclera. According to some embodiments, the method further comprises applying a composition comprising at least one plasminogen activator to at least one of said implant and said sclera following a desired time-period, thereby detaching said implant from said sclera; and removing said implant from said eye.

According to some embodiments, the present invention provides a method for reversibly affixing an implant to a sclera of an eye, the method comprising:
placing an implant on said sclera;
applying a composition comprising at least one fibrin-based tissue adhesive to at least one of said implant and said sclera, thereby affixing said implant to the sclera; and, following a desired time-period, applying a composition comprising at least one plasminogen activator to at least one of said implant and said sclera, thereby detaching said implant from said sclera; and removing said implant from said eye.

According to some embodiments, applying the composition comprising at least one fibrin-based tissue adhesive to at least one of the implant and the sclera is applying the composition on top of said implant. According to some embodiments, applying the composition comprising at least one fibrin-based tissue adhesive on top of the implant is configured to affix the implant to the sclera and to the conjunctiva of the eye. According to some embodiments, applying the composition comprising at least one fibrin-based tissue adhesive on top of said implant, is configured to form a layer of fibrin-glue which covers the implant (particularly the outer surface of the implant that is not in contact with, or facing, the sclera) and affixes the implant to both the sclera and conjunctiva.

According to some embodiments, the implant is a plaque. According to particular embodiments, the implant is a radioactive plaque. According to yet additional particular embodiments, the implant is a radioactive plaque made of a material which is able to block radiation emanating from within the plaque, such as, but not limited to gold. According to some embodiments, the implant is a gold plaque. According to some embodiments, the implant is a plaque made essentially of gold. As used herein, the term “essentially” refers to at least 80%, preferably at least 90%. Each possibility represents a separate embodiment of the present invention. According to other embodiments, the implant comprises radioactive seeds. According to some embodiments, the radioactive seeds comprise radioactive isotopes of elements selected from the group consisting of: Iodine, Cobalt, Ruthenium, Palladium, Radon and a combination thereof. Each possibility represents a separate embodiment of the present invention. According to some embodiments, the radioactive seeds are Iodine 125 seeds. According to some embodiments, the implant is a radioactive plaque comprising radioactive seeds. According to some embodiments, the implant comprises a radioactive source. According to some embodiments, the radioactive source is radioactive seeds.

According to some embodiments, the implant is a plaque comprising a protruding concentric annular rim. According to some embodiments, the implant is a radioactive plaque comprising a protruding concentric annular rim. Without wishing to be bound by any theory or mechanism, the protruding concentric annular rim is able to enhance the contact area between a plaque and the sclera. According to some embodiments, the composition comprising at least one fibrin-based tissue adhesive is configured to be positioned between the annular rim and the sclera. According to some embodiments, the protruding concentric annular rim of the radioactive plaque binds the sclera through the fibrin-based tissue adhesive. According to other embodiments, the fibrin-based tissue adhesive covers both the plaque and protruding concentric annular rim, thereby affixing the plaque to the sclera and conjunctiva. According to some embodiments, the protruding concentric annular rim is integrally formed with the plaque or radioactive plaque. Each possibility represents a separate embodiment of the present invention. According to some embodiments, the protruding concentric annular rim is made of a different material than the plaque or radioactive plaque. Each possibility represents a separate embodiment of the present invention. According to some embodiments, the plaque or radioactive plaque is made of an inert material, such as, but not limited to gold, whereas the protruding concentric annular rim is made of a material able to bind the fibrin glue, such as, but not limited to, plastic. Each possibility represents a separate embodiment of the present invention.

According to some embodiments, the implant comprises a pharmaceutical composition. According to some embodiments, the pharmaceutical composition comprises a therapeutic agent selected from the group consisting of: an antibiotic agent, an anti-inflammatory agent, a micronutrient, a cytotoxic agent, a chemotherapeutic agent, a cytokine, a growth factor and a combination thereof. Each possibility represents a separate embodiment of the present invention. According to some embodiments, the micronutrient is riboflavin. According to some embodiments, the implant comprises a pharmaceutical composition comprising riboflavin. According to some embodiments, a pharmaceutical composition comprising riboflavin is formulated as a slow release and/or sustained release composition. Each possibility represents a separate embodiment of the present invention.

According to some embodiments, the present invention provides a method of affixing an implant to a tissue, the method comprising placing said implant on said tissue and applying a composition comprising at least one fibrin-based tissue adhesive to at least one of said implant and said tissue, thereby affixing said implant to the tissue. According to some embodiments, the tissue is eye tissue, typically the sclera. According to some embodiments, the implant is a radioactive plaque. According to some embodiments, affixing using a composition comprising at least one fibrin-based tissue adhesive is configured to withstand a tension at least as high as the tension endured by an implant affixed by sutures.

As used herein, the terms “fibrin-based tissue adhesive”, “fibrin adhesive” and “fibrin glue” are used interchangeably and refer to an adhesive resulting from the reaction of at least fibrinogen and thrombin. According to some embodiments, the fibrin-based tissue adhesive is formed by mixing fibrinogen and thrombin found in separate compositions, either prior to or at the time of administration. Each possibility represents a separate embodiment of the present invention.

According to some embodiments, applying a composition comprising at least one fibrin-based tissue adhesive refers to applying the same composition to at least one of the implant and the tissue on which the implant is intended to be placed, such as the sclera. According to other embodiments, applying a composition comprising at least one fibrin-based tissue adhesive refers to applying separate compositions to at least one of the implant and the tissue on which the implant is intended to be placed, such as the sclera. According to some embodiments, compositions comprising the fibrin-based tissue adhesive comprise thrombin and fibrinogen in separate compositions.

According to some embodiments, applying a composition comprising at least one fibrin-based tissue adhesive comprises applying a composition comprising at least one component of said fibrin-based tissue adhesive. According to some embodiments, the at least one component of said fibrin-based tissue adhesive is selected from the group consisting of: fibrinogen, thrombin and a combination thereof.

According to some embodiments, placing an implant on a target tissue, such as the sclera, using a fibrin-based tissue adhesive is more accurate than placing the implant using sutures. According to some embodiments, affixing to the sclera according to the present invention comprises affixing to the area between the sclera and conjunctiva of the eye.

According to some embodiments, a desired time-period is a time-period sufficient for inducing a therapeutic effect, such as, but not limited to, providing a sufficient amount of radioactivity to treat an eye neoplasm such as uveal melanoma or to inhibit neovascularization in AMD or CNV patients. Each possibility represents a separate embodiment of the present invention. According to some embodiments, the desired time-period is between 1-7 days, typically between 2-5 days. Each possibility represents a separate embodiment of the present invention. According to some embodiments, a desired time period is determined by a treating physician according to clinical data.

According to some embodiments, following a desired time period, a composition comprising at least one plasminogen activator is applied to the site to which the fibrin-based tissue adhesive had been administered. According to some embodiments, the plasminogen activator is selected from the groups consisting of: tissue plasminogen activator, urokinase and a combination thereof. According to some embodiments, applying the composition comprising at least one plasminogen activator is configured to break down the fibrin-based tissue adhesive, by transforming endogenous plasminogen present in the eye tissue or exogenously-added plasminogen to the active enzyme plasmin, which in turn works to break down the fibrin-based tissue adhesive. Without wishing to be bound by any theory or mechanism, using a plasminogen activator breaks down at least part of the fibrin-based tissue adhesive to Fibrin Degradation Products (FDPs).

According to some embodiments, the present invention provides a method for breaking down an exogenous fibrin-based tissue adhesive in the body of a subject by administering a composition comprising at least one plasminogen activator to the site of said exogenous fibrin-based tissue adhesive in the subject's body.

According to some embodiments, the present invention provides a composition comprising at least one plasminogen activator for use in detaching an implant affixed to a tissue by a fibrin-based tissue adhesive. According to some embodiments, the present invention provides a composition comprising at least one plasminogen activator for use in detaching an implant affixed to a sclera of an eye by a fibrin-based tissue adhesive. According to some embodiments, the present invention provides a composition comprising at least one plasminogen activator for use in breaking down an exogenous fibrin-based tissue adhesive.

According to another aspect, the present invention provides a kit comprising: at least one composition comprising at least one fibrin-based tissue adhesive; a composition comprising at least one plasminogen activator; and an implant.

According to another aspect, the present invention provides a kit comprising: at least one composition comprising at least one fibrin-based tissue adhesive; and a composition comprising at least one plasminogen activator. According to some embodiments, the kit further comprises instructions of use thereof. According to some embodiments, the kit further comprises an implant. According to some embodiments, implant is a radioactive plaque. According to some embodiments, the kit of the invention is used to affix an implant to a tissue using exogenous fibrin-based tissue adhesive and remove the implant following a desired time period.

According to another aspect, the present invention provides a method of treating a medical condition in the eye of a subject in need thereof, the method comprising: placing a plaque on the sclera of said eye; and applying a composition comprising at least one fibrin-based tissue adhesive to at least one of: the sclera and said plaque, thereby affixing said radioactive plaque to the sclera. According to some embodiments, the method further comprises applying a composition comprising at least one plasminogen activator to at least one of said plaque and said sclera following a time period sufficient to treat said medical condition, thereby detaching said plaque from said sclera; and removing said plaque from said eye. According to some embodiments, applying a composition comprising at least one plasminogen activator is applying a composition between the sclera and conjunctiva at the site of an implant such as, but not limited to, a plaque.

According to some embodiments, the present invention provides a method of treating a neoplasm and/or CNV in the eye of a subject in need thereof, the method comprising:
placing a radioactive plaque on the sclera of said eye; and
applying a composition comprising at least one fibrin-based tissue adhesive to at least one of: the sclera and said radioactive plaque, thereby affixing said radioactive plaque to the sclera. Each possibility represents a separate embodiment of the present invention. According to some embodiments, the method further comprises applying a composition comprising at least one plasminogen activator to at least one of said radioactive plaque and said sclera following a time period sufficient to treat said neoplasm and/or CNV, thereby detaching said radioactive plaque from said sclera; and removing said radioactive plaque from said eye. Each possibility represents a separate embodiment of the present invention.

According to some embodiments, the present invention provides a method of treating a medical condition in the eye of a subject in need thereof, the method comprising:
placing a plaque on the sclera of said eye;
applying a composition comprising at least one fibrin-based tissue adhesive to at least one of: the sclera and said plaque, thereby affixing said plaque to the sclera; and, following a time-period sufficient to treat said medical condition
applying a composition comprising at least one plasminogen activator to at least one of said plaque and said sclera, thereby detaching said plaque from said sclera; and
removing said plaque from said eye.

According to some embodiments, the medical condition is selected from the group consisting of: an eye neoplasm, choroidal neovascularization, myopia and a combination thereof. Each possibility represents a separate embodiment of the present invention. According to some embodiments, choroidal neovascularization refers to a disease or disorder comprising CNV, such as, but not limited to, AMD.

According to some embodiments, the medical condition is selected from the group consisting of: an eye neoplasm, CNV and a combination thereof. Each possibility represents a separate embodiment of the present invention. According to some embodiments, the plaque is a radioactive plaque. According to some embodiments, the medical condition is an eye neoplasm and/or CNV and the plaque is a radioactive plaque. Each possibility represents a separate embodiment of the present invention.

According to some embodiments, the eye neoplasm is selected from the group consisting of: malignant melanoma, intraocular lymphoma, retinoblastoma, medulloepithelioma, intraocular metastasis, CHRPE (congenital hyperplasia of the retinal pigment epithelium), hemangioma and a combination thereof. Each possibility represents a separate embodiment of the present invention. According to some embodiments, the eye neoplasm is uveal melanoma. According to some embodiments, the plaque comprises a protruding concentric annular rim. According to some embodiments, the radioactive plaque comprises a protruding concentric annular rim. According to some embodiments, the composition comprising at least one fibrin-based tissue adhesive is configured to be positioned between the annular rim and the sclera.

According to some embodiments, the medical condition is myopia. According to some embodiments, the plaque comprises a pharmaceutical composition. According to some embodiments, the plaque comprises a pharmaceutical composition comprising riboflavin. According to some embodiments, the medical condition is myopia and the plaque comprises a pharmaceutical composition comprising riboflavin. According to some embodiments, the method of treating myopic comprises irradiating the sclera with ultra violet radiation.

Further embodiments, features, advantages and the full scope of applicability of the present invention will become apparent from the detailed description and drawings given hereinafter. However, it should be understood that the detailed description, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1 is a photograph, demonstrating, according to some embodiments of the invention, application of fibrin-glue components to a gold plaque placed on the sclera of an extracted porcine eye. The plaque is placed between the sclera and conjunctiva and the fibrin glue components are applied on the side of the plaque facing the conjunctiva.

FIG. 2 is a photograph, demonstrating, according to some embodiments of the invention, measurement of adhesion strength of a gold plaque affixed to the sclera of an extracted porcine eye. The adhesion strength was measured using a system comprising a tension transducer, a pulley, a transmission wire and an electrical winch.

FIG. 3A is an illustration, demonstrating, according to some embodiments of the invention, a gold plaque (100) having a concentric annular rim (102).

FIG. 3B is an illustration, demonstrating a concentric annular rim (102) which may be included in a plaque that is intended for placement on the sclera, according to certain embodiments of the invention.

FIG. 4A depicts, according to some embodiments of the invention, an implant (406) placed between the sclera (402) and conjunctiva (404) of an eye, prior to application of a composition comprising fibrin-based tissue adhesive.

FIG. 4B depicts, according to some embodiments of the invention, application of a composition comprising fibrin-based tissue adhesive (408) on top of an implant (406) placed between the sclera (402) and conjunctiva (404) of an eye.

### DETAILED DESCRIPTION OF THE INVENTION

The present invention provides according to some aspects methods and kits for reversibly affixing an implant, such as a radioactive plaque as a non-limiting example, to the sclera of an eye of subject. The subject according to embodiments of the present invention is a mammal, typically a human.

As exemplified herein below, affixing an implant to the sclera of a subject according to methods presented herein was advantageously found to be safe with regard to ocular surface tissue reaction and to cause no elevation in intraocular pressure. In addition, the implants show negligible horizontal movement and no tilting, thus facilitating treatment success. With respect to radioactive plaques in particular, radioactivity advantageously has no impact or weakening effect on the adhesive properties of the biological glue, nor does the glue absorb radiation to cause its attenuation, as exemplified hereinbelow.

### Definitions

As used herein the term “implant” refers to an element or device configured for insertion into the body. An implant according to embodiments of the present invention is essentially not a living tissue implant Implants according to embodiments of the present invention are typically configured for temporary use, namely, configured for insertion into the body for a desired period of time, after which they can be removed. According to some embodiments, the implant is a non-tissue derived implant. According to some embodiments, the implant is an artificial implant. According to some embodiments, the implant is a metal or metal alloy implant. Each possibility represents a separate embodiment of the present invention. According to some embodiments, an implant, as referred to herein, is made of a non-organic material, such as, but not limited to: a metal, a synthetic polymer, a silicone, a silicone derivative or a combination thereof. Each possibility represents a separate embodiment of the present invention. According to some embodiments, a synthetic polymer is a plastic polymer. According to some embodiments, an implant, as referred to herein, is made of organic plastic. According to some embodiments, the implant is a synthetic implant. According to some embodiments, a synthetic implant is composed of a non-organic polymer. According to some embodiments, the implant is biocompatible. According to some embodiments, the metal is gold. According to some embodiments, the implant is a gold implant. According to some embodiments, the implant is a gold plaque.

According to some embodiments, the implant is a plaque. According to some embodiments, the implant is a gold plaque. According to some embodiments, the implant is a radioactive plaque. As used herein, a plaque refers to a convex device configured to form a sealed space when placed on a surface. According to some embodiments, the plaque comprises a pharmaceutical composition and/or a radioactive source located within the sealed space and faces said surface. Each possibility represents a separate embodiment of the present invention. As used herein, a radioactive plaque refers to a convex device configured to form a sealed space when placed on a surface, wherein the plaque comprises a radioactive source located within the sealed space and faces said surface. According to some embodiments, the surface is the sclera of an eye. According to some embodiments, the implant is a radioactive plaque made of gold, comprising radioactive seeds.

According to some embodiments, the implant comprises a radioactive source. According to some embodiments, an implant is a radioactive plaque. According to some embodiments, the radioactive source is radioactive seeds. According to some embodiments, the radioactive source is radioactive seeds embedded in a surface, such as, but not limited to, a silicone surface. According to some embodiments, the radioactive seeds comprise Iodine-125. According to some embodiments, the radioactive plaque is configured to match the convexity of the sclera. According to some embodiments, the radioactive plaque is composed of gold. According to some embodiments, the radioactive plaque is made of a material and/or structure which are configured to prevent the spread of radioactivity outside of the sealed space formed by the plaque. Each possibility represents a separate embodiment of the present invention.

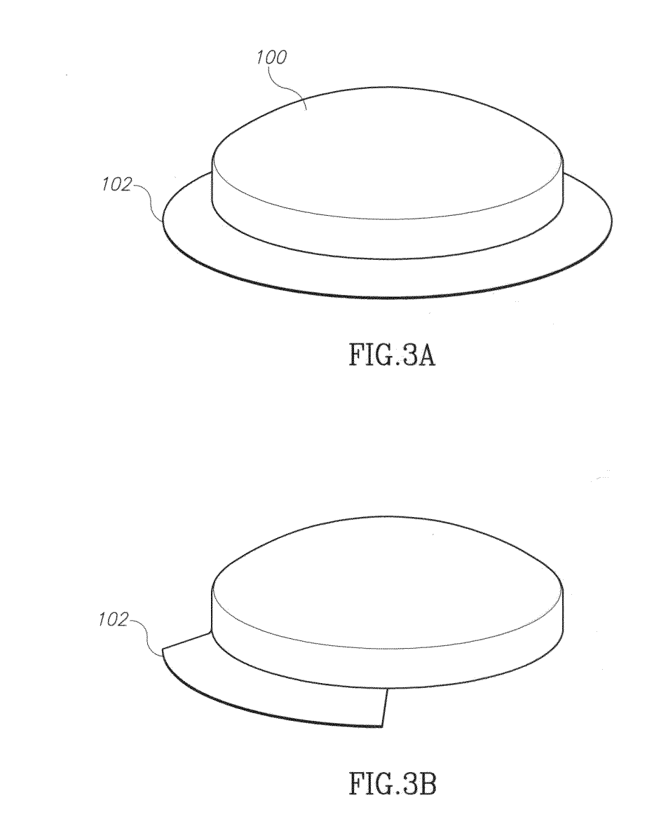

Reference is now made to FIG. 3A, which depicts a perspective side view of radioactive plaque (100) having a concentric annular rim (102), according to some embodiments of the invention. Of note, FIG. 3A depicts only a sector of concentric annular rim (102). According to some embodiments, an implant, such as, but not limited to, a plaque or a radioactive plaque, comprises a protruding concentric annular rim. Each possibility represents a separate embodiment of the present invention. According to some embodiments, a protruding concentric annular rim refers to a rim which protrudes radially outwardly around the implant, from an edge of the implant that is configured to face the sclera, and is configured to be in contact with the surface on which the implant is placed. According to some embodiments, the protruding concentric annular rim is essentially convex. According to some embodiments, the protruding concentric annular rim is essentially convex such that it is configured to be placed on the surface of a sclera. According to some embodiments, the protruding concentric annular rim surrounds the entire circumference or edge of the implant. According to some embodiments, the protruding concentric annular rim of a radioactive plaque is fully circular and surrounds the edge of the plaque configured to touch the sclera. According to other embodiments, the protruding concentric annular rim surrounds a part of the circumference or edge of the implant, such that it forms, in a non-limiting example, a sector of a circle. According to some embodiments, such as the embodiment presented in FIG. 3B, the protruding concentric annular rim is a circular sector protruding from at least part of the edge of the radioactive plaque configured to touch the sclera. According to some embodiments, the protruding concentric annular rim is composed of a different material than the implant. According to certain embodiments, the protruding concentric annular rim is composed of the same material that the implant is made of. According to some embodiments, the protruding concentric annular rim is made of plastic. According to some embodiments, the implant is a radioactive plaque made of gold, comprising a plastic protruding concentric annular rim. According to some embodiments, the protruding concentric annular rim is integrally formed with the implant.

According to some embodiments, the composition comprising at least one fibrin-based tissue adhesive is configured to be positioned between the protruding concentric annular rim and the sclera. Without wishing to be bound by any theory or mechanism, an implant having a protruding concentric annular rim is able to attach to a sclera through fibrin glue adhering to the rim with a high adhesion strength.

As noted above, the terms “fibrin-based tissue adhesive”, “fibrin adhesive” and “fibrin glue”, as used herein, are used interchangeably and refer to an adhesive composed mainly of fibrin resulting from the reaction of at least fibrinogen and thrombin. As used herein, a fibrin glue refers to an exogenous fibrin glue. As used herein, an exogenous fibrin glue is a fibrin glue formed through reaction of at least one exogenous component. As used herein, the term an exogenous component refers to a component which did not originate in the body of a subject which the fibrin glue is administered to. According to some embodiment, a fibrin glue is formed by the reaction of exogenous fibrinogen and exogenous thrombin. Fibrinogen is a glycoprotein that is converted to fibrin by the serine protease Thrombin, thus forming a fibrin clot which can be used as a glue or sealant. According to some embodiments, fibrin glue may further comprise at least one excipient selected from the group consisting of: fibronectin, plasminogen, Factor XIII, aprotinin, CaCl₂ and a combination thereof. Each possibility represents a separate embodiment of the present invention.

According to some embodiments, the fibrin-based tissue adhesive is formed by mixing fibrinogen and thrombin found in separate compositions, either prior to or at the time of administration. Each possibility represents a separate embodiment of the present invention. According to some embodiments, the fibrin-based tissue adhesive is formed by mixing a solution comprising at least thrombin and CaCl₂ with a solution comprising at least fibrinogen and possibly further comprising aprotinin, Factor XIII and plasminogen. Each possibility represents a separate embodiment of the present invention. Non-limiting examples of fibrin-based tissue adhesives include the commercially available products TISSEEL® (Baxter Health Corporation) and EVICEL® (Johnson & Johnson).

According to some embodiments, applying a composition comprising at least one fibrin-based tissue adhesive refers to applying a composition comprising at least thrombin and fibrinogen. According to some embodiments, applying a composition comprising at least one fibrin-based tissue adhesive refers to applying a composition comprising at least one component of said fibrin-based tissue adhesive. According to some embodiments, a component of the fibrin-based tissue adhesive is selected from the group consisting of: fibrinogen and thrombin. According to some embodiments, applying a composition comprising fibrin glue in order to glue two surfaces comprises applying a composition comprising at least fibrinogen to the first surface to be glued and applying a composition comprising at least thrombin to the second surface to be glued. In a non-limiting example, applying a composition comprising a fibrin-based tissue adhesive to at least one of an implant and a sclera comprises applying a composition comprising thrombin to the sclera and a composition comprising fibrinogen to the implant, or vice versa. Each possibility represents a separate embodiment of the present invention. According to some embodiments, applying a composition comprising a fibrin-based tissue adhesive to at least one of an implant and a sclera comprises applying a composition comprising fibrinogen and a composition comprising thrombin to the implant, to the sclera or to both. Each possibility represents a separate embodiment of the present invention.

According to some embodiments, applying a composition comprising a fibrin glue in order to glue two surfaces comprises applying the fibrin glue between said surfaces. According to some embodiments, applying a composition comprising a fibrin glue in order to glue an implant to a sclera comprises applying the fibrin glue between the implant and the sclera.

According to some embodiments, applying a composition comprising a fibrin glue in order to glue an implant to a sclera comprises applying the fibrin glue on the implant after it has been placed on the sclera. According to some embodiments, applying a composition comprising a fibrin glue in order to glue an implant to a sclera comprises applying the fibrin glue on the implant after it has been placed on the sclera, thus affixing the implant to the sclera and the conjunctiva. According to some embodiments, applying a composition comprising fibrin glue on an implant comprises forming a layer of fibrin glue covering at least part of said implant and at least part of the sclera. According to some embodiments, the layer of fibrin glue affixes the implant to the sclera. According to other embodiments, the layer of fibrin glue affixes the implant to the sclera and overlying conjunctiva.

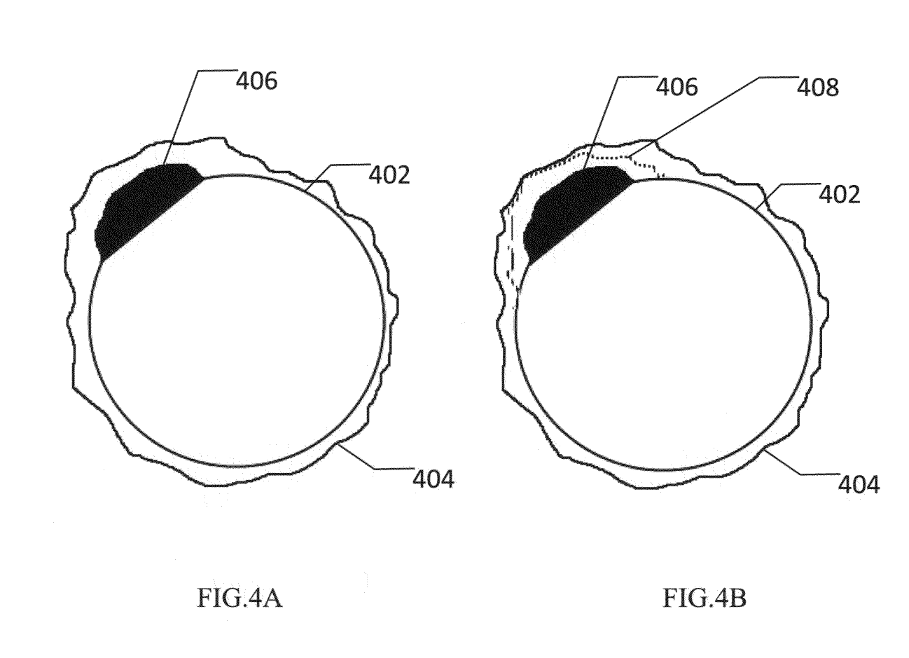

Reference is now made to FIGS. 4A and 4B, which schematically depict application of a composition comprising fibrin glue according to some embodiments of the present invention. An implant (406) is first placed between the sclera (402) and conjunctiva (404) of an eye, followed by application of a composition comprising fibrin-based tissue adhesive (408) on top of the implant (406). As demonstrated in FIG. 4B, the composition comprising fibrin-based tissue adhesive (408) forms a layer of fibrin glue which covers the implant (406) and part of the sclera (402). The fibrin glue covering the implant (406), formed by application of the composition comprising fibrin-based tissue adhesive (408), adheres both to the sclera (402) and the overlying conjunctiva (404).

According to some embodiments, applying a composition comprising fibrin glue in order to glue an implant to a sclera comprises applying the fibrin glue in the implant's vicinity in between the sclera and conjunctiva. According to some embodiments, applying a composition comprising fibrin glue in order to glue an implant to a sclera comprises applying the fibrin glue such that it covers at least part of the implant and part of the sclera. Without wishing to be bound by any theory or mechanism, applying a composition comprising fibrin glue on top of an implant placed on the sclera enables to strongly affix the implant in place as it adheres both the sclera and overlying conjunctiva. According to some embodiments, applying a composition comprising fibrin glue on top of an implant placed on the sclera enables strongly affixing an implant which is inert to fibrin glue to the sclera and conjunctiva.

As used herein, the term “plasminogen activator” refers to a protease which is able to convert plasminogen to plasmin According to some embodiments, a plasminogen activator converts plasminogen to plasmin which in turn promotes breaking down of a fibrin clot (also referred to herein as fibrinolysis). It is to be understood that plasma constituents comprising at least plasminogen are required in order for a plasminogen activator to promote fibrinolysis of a fibrin clot. Without being bound by any theory or mechanism, applying a composition comprising at least one plasminogen activator to a fibrin clot formed through the use of a fibrin adhesive results in fibrinolysis of the fibrin clot, enabling detachment of the surfaced glued by the adhesive.

According to some embodiments, the plasminogen activator is selected from the group consisting of: tissue plasminogen activator, urokinase and a combination thereof. Each possibility represents a separate embodiment of the present invention. According to some embodiments, a composition comprising at least one plasminogen activator further comprises CaCl₂.

According to some embodiments, applying the composition comprising at least one plasminogen activator is configured to break down a fibrin-based tissue adhesive. According to some embodiments, applying the composition comprising at least one plasminogen activator is configured to break down a fibrin-based tissue adhesive thus enabling detachment of the surfaces glued by said adhesive. According to some embodiments, applying a composition comprising at least one plasminogen activator is applying said composition between the sclera and conjunctiva at the site of the implant. According to some embodiments, applying a composition comprising at least one plasminogen activator between the sclera and conjunctiva at the site of the implant induces breaking of the layer of fibrin glue affixing the implant to the sclera and conjunctiva.

As used herein, the term “affixing” refers to attaching in a manner which essentially prevents movement of the attached object. The tension force required to detach an affixed object is referred to herein as adhesion strength.

The terms “comprises”, “comprising”, “includes”, “including”, “having” and their conjugates mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. In the description and claims of the application, each of the words “comprise” “include” and “have”, and forms thereof, are not necessarily limited to members in a list with which the words may be associated.

The term “about” when referring to a measurable value such as amount, temporal duration, and the like, is meant to encompass variations of +/−10%, 15 more preferably +/−5%, even more preferably +/−1%, and still more preferably +/−0.1% from the specified value, as such variations are appropriate to perform the disclosed methods.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to plus/minus 10% of the value stated.

### Embodiments of the invention

According to some embodiments, the present invention provides for the first time a composition comprising at least one plasminogen activator for breaking down a fibrin clot formed by exogenous fibrin glue. According to some embodiments, the present invention provides a method for affixing an implant to an eye of a subject using exogenous fibrin glue. According to some embodiments, the present invention provides a method for affixing an implant to the sclera of an eye using exogenous fibrin glue, the method comprising administration of fibrin glue on top of an implant placed on the sclera such that a layer of fibrin is formed and affixes at least part of the implant to the sclera and overlying conjunctiva. According to some embodiments, the present invention provides a method for reversibly affixing an implant to an eye of a subject using exogenous fibrin glue, wherein the exogenous fibrin glue is broken down using a composition comprising at least one plasminogen activator, thus enabling detachment of the affixed implant.

According to one aspect, the present invention provides a method (referred to herein as the method of the invention) for reversibly affixing an implant to a sclera of an eye, the method comprising:
placing an implant on said sclera; and applying a composition comprising at least one fibrin-based tissue adhesive to at least one of said implant and said sclera, thereby affixing said implant to the sclera. According to some embodiments, applying a composition comprising at least one fibrin-based tissue adhesive to at least one of said implant and said sclera refers to applying said composition over said implant, such that it covers at least part of the implant and at least part of the sclera. According to some embodiments, applying a composition comprising at least one fibrin-based tissue adhesive to at least one of said implant and said sclera refers to applying said composition over said implant, such that it covers at least part of the implant and adheres to both the sclera and overlying conjunctiva.

According to some embodiments, the method of the invention further comprises applying a composition comprising at least one plasminogen activator to at least one of said implant and said sclera following a desired time-period, thereby detaching said implant from said sclera; and removing said implant from the eye. According to some embodiments, applying a composition comprising at least one plasminogen activator to at least one of said implant and said sclera refers to applying said composition between the sclera and overlying conjunctiva at the site of the implant.

According to some embodiments, the present invention provides a method for reversibly affixing an implant to a sclera of an eye, the method comprising:
placing an implant on said sclera;
applying a composition comprising at least one fibrin-based tissue adhesive to at least one of said implant and said sclera, thereby affixing said implant to the sclera; and, following a desired time-period, applying a composition comprising at least one plasminogen activator to at least one of said implant and said sclera, thereby detaching said implant from said sclera; and removing said implant from the eye. According to some embodiments, the implant is a plaque. According to some embodiments, the implant is a radioactive plaque.

According to some embodiments, the present invention provides a method for reversibly affixing a radioactive plaque to a sclera of an eye, the method comprising:
placing a radioactive plaque on said sclera;
applying a composition comprising at least one fibrin-based tissue adhesive to at least one of said radioactive plaque and said sclera, thereby affixing said radioactive plaque to the sclera; and, following a desired time-period, applying a composition comprising at least one plasminogen activator to at least one of said radioactive plaque and said sclera, thereby detaching said radioactive plaque from said sclera; and removing the radioactive plaque from the eye.

According to some embodiments, placing an implant on the sclera refers to placing the implant between the sclera and conjunctiva. According to some embodiments, placing an implant on the sclera refers to placing at the site to be treated by the implant. According to some embodiments, placing an implant on the sclera refers to placing at a site of a neoplasm. According to some embodiments, placing an implant on the sclera refers to placing at the site of uveal melanoma. According to some embodiments, placing an implant on the sclera refers to placing at a site of CNV. According to some embodiments, placing an implant on the sclera refers to placing at a site of AMD. According to some embodiments, placing an implant on the sclera refers to placing a radioactive plaque at a site of uveal melanoma. According to some embodiments, placing an implant on the sclera refers to placing a radioactive plaque at a site of AMD. According to some embodiments, placing an implant on the sclera refers to placing a radioactive plaque at a site of CNV.

According to some embodiments, a composition comprising at least one fibrin-based tissue adhesive comprises a single fibrin-based adhesive. According to some embodiments, a composition comprising at least one fibrin-based tissue adhesive comprises a plurality of fibrin-based tissue adhesives. As used herein, the term plurality refers to at least two. According to some embodiments, a composition comprising at least one fibrin-based tissue adhesive comprises a plurality of fibrin-based tissue adhesives which differ in the excipients comprised in said fibrin-based tissue adhesive. According to some embodiments, the fibrin-based tissue adhesive comprises at least one excipient. According to some embodiments, the fibrin-based tissue adhesive comprises at least one excipient selected from the group consisting of: fibronectin, plasminogen, Factor XIII, aprotinin, CaCl₂ and a combination thereof. Each possibility represents a separate embodiment of the present invention.

According to some embodiments, the method of the invention provides fixation of at least one surface of an implant to at least part of the surface of the sclera. According to some embodiments, the fixation of the implant to the sclera is achieved by applying a composition comprising at least one exogenous fibrin-based adhesive to at least one of the sclera and implant. Each possibility represents a separate embodiment of the present invention. According to some embodiments, the fixation of the implant to the sclera is achieved by applying a composition comprising at least one exogenous fibrin-based adhesive over the implant such that a layer of fibrin glue covers at least part of the implant and at least part of the sclera. Without wishing to be bound by any theory or mechanism, affixing an implant to the sclera using a composition comprising fibrin glue provides for: (a) accurate placement of the implant on the sclera, (b) fixation to the sclera having a high adhesion strength, (c) fixing the implant both to the sclera and overlying conjunctiva, and (d) easily detaching and removing the implant by breaking down the fibrin glue using a composition comprising a plasminogen activator.

According to some embodiments, the composition comprising at least one fibrin-based adhesive is applied to at least one of the implant and the sclera prior to placing the implant on the sclera. According to other embodiments, the composition comprising at least one fibrin-based adhesive is applied to at least one of the implant and the sclera after placing the implant on the sclera. According to some embodiments, the implant is placed on the surface of the sclera, in between the sclera and the conjunctiva.

According to some embodiments, the composition comprising at least one fibrin-based tissue adhesive is first applied to at least part of the surfaces of the implant and/or the sclera in which fixation is desired and then the implant is placed on the sclera. Each possibility represents a separate embodiment of the present invention. According to other embodiments, the implant is first placed on the sclera and then the composition comprising at least one fibrin-based tissue adhesive is applied to at least part of the surfaces of the implant and/or the sclera in which fixation is desired. Each possibility represents a separate embodiment of the present invention. According to some embodiments, the implant is first placed on the surface of the sclera and then the composition comprising at least one fibrin-based tissue adhesive is applied on top of the implant, such that it forms a layer of fibrin glue covering at least part of the implant and at least part of the sclera. According to some embodiments, the implant is first placed on the surface of the sclera and then the composition comprising at least one fibrin-based tissue adhesive is applied between the sclera and conjunctiva at the area of the implant, such that a layer of fibrin glue is formed, covering at least part of the implant and at least part of the sclera. According to some embodiments, the composition comprising at least one fibrin-based tissue adhesive is applied between the conjunctiva and the implant placed on the sclera, such that a layer of fibrin glue is formed, covering at least part of the implant and at least part of the sclera.

According to some embodiments, applying a composition comprising at least one fibrin-based tissue adhesive comprises applying a composition comprising at least one component of said fibrin-based tissue adhesive. According to some embodiments, a component of a fibrin-based tissue adhesive is selected from the group consisting of: fibrinogen and thrombin. According to some embodiments, applying a composition comprising at least one fibrin-based tissue adhesive comprises applying a composition comprising fibrinogen and a separate composition comprising thrombin. According to some embodiments, applying a composition comprising a fibrin-based tissue adhesive comprises applying at least one component of the fibrin-based tissue adhesive to the implant and at least another component of the fibrin-based tissue adhesive to the sclera. According to some embodiments, applying a composition comprising a fibrin-based tissue adhesive comprises applying a composition comprising at least fibrinogen to the implant and applying a composition comprising at least thrombin to the sclera, or vice versa. Each possibility represents a separate embodiment of the present invention.

According to some embodiments, an implant affixed to a sclera using a composition comprising at least one fibrin-based tissue adhesive has an adhesion strength at least as high as an implant affixed to a sclera using sutures. According to some embodiments, an implant affixed to a sclera using a composition comprising at least one fibrin-based tissue adhesive has higher adhesion strength than an implant affixed to a sclera using sutures. As exemplified herein below, the method of the invention enables affixing an implant to a sclera such that the implant remains in place for the desired time period.

According to the method of the invention, the implant remains affixed to the sclera for a time period sufficient for treatment administered by said implant. According to some embodiments, the desired time period is determined by a treating physician. According to some embodiments, the desired time-period is variable and depends on the treated patient and/or treated condition. According to some embodiments, the desired time period is about 1-7 days, typically about 2-5 days. Each possibility represents a separate embodiment of the present invention. According to some embodiments, a desired time period is a time period sufficient for treating a medical condition selected from the group consisting of: an eye neoplasm, age-related macular degeneration, myopia and a combination thereof. Each possibility represents a separate embodiment of the present invention. According to some embodiments, a desired time period is a time period sufficient for treating an eye neoplasm and/or AMD and/or CNV using a radioactive plaque. Each possibility represents a separate embodiment of the present invention. According to some embodiments, a desired time period is a time period sufficient for treating uveal melanoma using a radioactive plaque. According to some embodiments, a time period sufficient to treat uveal melanoma using a radioactive plaque is about 1-7 days, typically about 2-5 days. Each possibility represents a separate embodiment of the present invention.

According to some embodiments, a composition comprising at least one plasminogen activator according to the method of the invention comprises a sufficient amount of plasminogen activator to enable breaking down at least part of the exogenous fibrin glue used to affix the implant to the sclera. According to some embodiments, applying a composition comprising at least one plasminogen activator enables to break down at least part of the exogenous fibrin glue used to affix the implant to the sclera, thus enabling to detach the implant from the sclera. According to some embodiments, applying a composition comprising at least one plasminogen activator comprises applying between the sclera and conjunctiva, at the site of the implant. According to some embodiments, the concentration of plasminogen activator within the composition comprising at least one plasminogen activator is between about 100-10,000 Units/ml. According to some embodiments, the concentration of plasminogen activator within the composition comprising at least one plasminogen activator is about 1000 Units/ml.

According to some embodiments, the present invention provides a composition comprising at least one plasminogen activator for use in detaching an implant affixed to the sclera of an eye through at least one fibrin-based tissue adhesive.

According to another aspect, the present invention provides a kit (referred to herein as the kit of the invention) comprising: at least one composition comprising at least one fibrin-based tissue adhesive; and a composition comprising at least one plasminogen activator. According to some embodiments, the kit of the invention further comprises an implant. According to some embodiments, the kit of the invention further comprises a plaque. According to some embodiments, the kit of the invention further comprises a radioactive plaque.

According to some embodiments, the present invention provides a kit comprising: at least one composition comprising at least one fibrin-based tissue adhesive; a composition comprising at least one plasminogen activator; and a plaque. According to some embodiments, the present invention provides a kit comprising: at least one composition comprising at least one fibrin-based tissue adhesive; a composition comprising at least one plasminogen activator; and a radioactive plaque.

According to some embodiments, the fibrin adhesive within the kit of the invention comprises at least two compositions, one comprising at least fibrinogen and another comprising at least thrombin. According to some embodiments, the kit of the invention comprises at least one composition comprising at least one fibrin-based tissue adhesive or a component thereof and a composition comprising at least one plasminogen activator. According to some embodiments, the kit further comprises instructions of use thereof.

According to some embodiments, the kit of the invention is used to affix an implant to a tissue using exogenous fibrin-based tissue adhesive and remove the implant following a desired time period. According to some embodiments, the present invention provides the kit of the invention for use in reversibly affixing an implant to a sclera of an eye. According to some embodiments, the present invention provides the kit of the invention for use in reversibly affixing an implant to a sclera of an eye, wherein the at least one composition comprising at least one fibrin-based tissue adhesive is configured to enable affixing said implant to the sclera; and wherein the composition comprising at least one plasminogen activator is configured to enable detachment of the implant from the sclera. According to some embodiments, the kit further comprises an implant. According to some embodiments, the kit further comprises a radioactive plaque.

According to some embodiments, the present invention provides a method for breaking down an exogenous fibrin-based tissue adhesive in the body of a subject by administering a composition comprising at least one plasminogen activator to the site of said exogenous fibrin-based tissue adhesive in the subject's body. According to some embodiments, the present invention provides a composition comprising at least one plasminogen activator for use in breaking down an exogenous fibrin-based tissue adhesive. According to some embodiments, the present invention provides for the first time kits and methods for reversible attachment using fibrin glue.

The present invention further provides, according to further embodiments, a method of treating a medical condition in the eye. According to some embodiments, the medical condition of the eye is a scleral degenerative process. According to some embodiments, the medical condition of the eye is a macular disease. According to some embodiments, the medical condition of the eye is a disease or pathology of the posterior segment of the eye. According to some embodiments, the medical condition of the eye is selected from the group consisting of: an eye neoplasm, choroidal neovascularization (CNV), myopia and a combination thereof. Each possibility represents a separate embodiment of the present invention. According to some embodiments, the medical condition of the eye is selected from the group consisting of: an eye neoplasm, choroidal neovascularization (CNV), myopia, scleral degenerative diseases and a combination thereof. Each possibility represents a separate embodiment of the present invention. According to some embodiments, treating CNV comprises treating a disease or disorder comprising CNV. According to some embodiments, treating CNV comprises treating AMD.

According to some embodiments, the present invention provides a method of treating a medical condition in the eye of a subject in need thereof, the method comprising affixing an implant comprising a pharmaceutical composition comprising at least one therapeutic agent to the sclera of said subject by applying a composition comprising at least one fibrin-based tissue adhesive to at least part of said implant and at least part of said sclera.

According to one aspect, the present invention provides a method of treating a medical condition in the eye of a subject in need thereof, the method comprising:
placing a plaque on the sclera of said eye and applying a composition comprising at least one fibrin-based tissue adhesive to at least one of: the sclera and said plaque, thereby affixing said plaque to the sclera. According to some embodiments, the method further comprises applying a composition comprising at least one plasminogen activator to at least one of said plaque and said sclera following a time-period sufficient to treat said medical condition, thereby detaching said plaque from said sclera; and removing said plaque from said eye.

According to some embodiments, the present invention provides a method of treating a medical condition in the eye of a subject in need thereof, the method comprising:
placing a plaque on the sclera of said eye;
applying a composition comprising at least one fibrin-based tissue adhesive to at least one of: the sclera and said plaque, thereby affixing said plaque to the sclera; and, following a time-period sufficient to treat said medical condition applying a composition comprising at least one plasminogen activator to at least one of said plaque and said sclera, thereby detaching said plaque from said sclera; and removing said plaque from said eye.

According to some embodiments, the present invention provides a method of treating a medical condition selected from the group consisting of: uveal melanoma, CNV and a combination thereof in the eye of a subject in need thereof, the method comprising:
placing a radioactive plaque on the sclera of said eye;
applying a composition comprising at least one fibrin-based tissue adhesive to at least one of: the sclera and said radioactive plaque, thereby affixing said radioactive plaque to the sclera; and, following a time-period sufficient to treat said medical condition applying a composition comprising at least one plasminogen activator to at least one of said radioactive plaque and said sclera, thereby detaching said radioactive plaque from said sclera; and
removing said radioactive plaque from said eye.

According to some embodiments, treatment of CNV using a radioactive plaque according to the present invention comprises inhibiting neovascularization. According to some embodiments, treatment of AMD using a radioactive plaque according to the present invention comprises inhibiting neovascularization.

According to some embodiments, the present invention provides a method for treating myopia, the method comprising placing a plaque on the sclera of said eye, wherein said plaque comprises a composition comprising riboflavin; and applying a composition comprising at least one fibrin-based tissue adhesive to at least one of: the sclera and said plaque, thereby affixing said plaque to the sclera. According to some embodiments, the method further comprises applying a composition comprising at least one plasminogen activator to at least one of said plaque and said sclera following a time-period sufficient to treat said myopia, thereby detaching said plaque from said sclera; and removing said plaque from said eye.

According to some embodiments, the present invention provides a kit comprising:
at least one composition comprising at least one fibrin-based tissue adhesive;
a composition comprising at least one plasminogen activator; and
a radioactive plaque
for the treatment of a neoplasm in the eye of a subject.

According to some embodiments, the present invention provides a kit comprising:
at least one composition comprising at least one fibrin-based tissue adhesive;
a composition comprising at least one plasminogen activator; and
a radioactive plaque
for the treatment of uveal melanoma in the eye of a subject.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Fibrin Glue is Able to Strongly Affix a Plaque to the Sclera of an Eye in-vitro

In order to test whether fibrin glue may be used for affixing a plaque to the sclera of an eye, extracted porcine eyes were used. The porcine eyes were enucleated from 6 month old (weight ˜90 Kg) domesticated pigs. The eyes were eviscerated through an 180°-270° corneo-limbal incision, filled with glass beads and sutured with a 5/0 prolen suture in order to maintain a constant intraocular pressure. The eyes were then anchored to a stainless steel perforated cover using 3/0 silk sutures (full thickness through the conjunctiva and sclera). The cover was attached to a vertical stand to simulate a supine lying patient.

In five of the examined eyes, 12 mm COMS-style gold plaques without radioactive seeds (Eckert & Ziegler BEBIG, Berlin, Germany) were affixed to the sclera using 5/0 nylon sutures through eyelets 1, 3 and 5 followed by closure of the conjunctiva using vicryl 7/0 sutures, as known in the art for affixing radioactive plaques. Before positioning the plaques in place, two 4/0 silk sutures were passed through 2 of the plaque eyelets (number 2 and 4) and were tied to create 2 small loops. When placing the plaques, these loops were positioned facing the limbus end. These loops were later used to connect the fibrin glue-plaque-eye wall complex to an adhesion strength-measuring device.

In five additional eyes similar gold plaques were placed on the sclera of each eye, beneath the conjunctiva, as follows:

Through a 120° limbal base incision, the conjunctiva and Tenon's capsule were cautiously under-dissected and a 12 mm COMS-style gold plaques (Eckert & Ziegler BEBIG, Berlin, Germany) containing a silicone mold without any radioactive seeds were placed 4-6 mm posterior to the limbus of each eye, on the sclera and beneath the conjunctiva and Tenon's capsule. Before positioning the plaques in place, two 4/0 silk sutures were passed through 2 of the plaque eyelets (number 2 and 4) and were tied to create 2 small loops. When placing the plaques, these loops were positioned facing the limbus end. Next, Two ml fibrin glue set (Tisseel®, Baxter Corporation, Mississauga, Canada) was prepared according to the manufacturer's guidelines and the glue components were injected above and on the immediate surroundings of the plaque, but not beneath it. During the injection the conjunctiva was held in suspense above the plaque using forceps and at the end of the process the anterior aspect of the conjunctiva was placed in proximity to its limbal insertion.

In yet other five additional eyes, the conjunctiva and Tenon's capsule were dissected and removed, so only bare sclera remained, and gold plaques were affixed to the sclera using fibrin glue as described above.

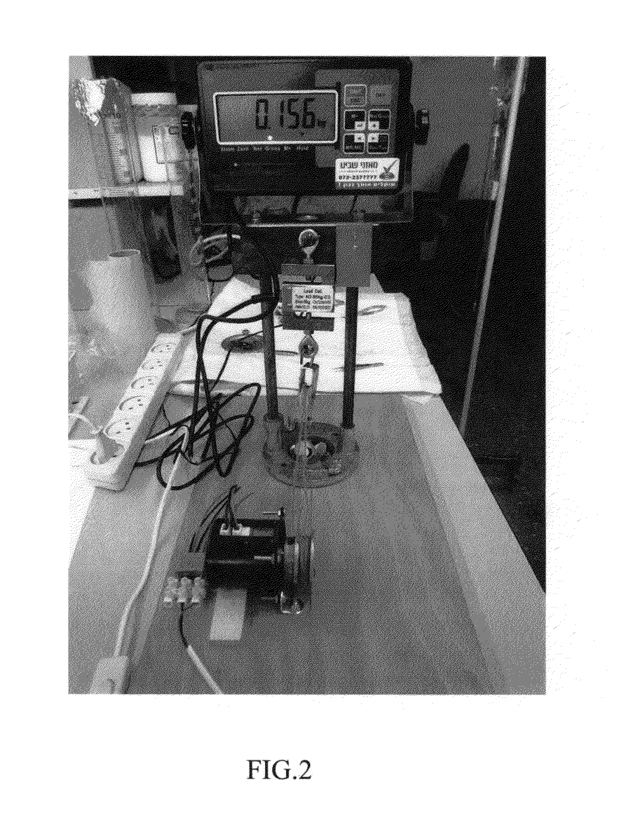

After affixing the gold plaques to each eye, the eyes treated with fibrin glue were incubated in plasma (MDAIS, Ramat Gan, Israel) for 5 days (120 hours) to simulate a fibrinolytic environment (the plasma was replaced every 36 hours). The adhesion strength of the affixed plaques in all groups was then measured using a system constructed of a tension transducer, a pulley, a transmission wire and an electrical winch (FIG. 2). The system included a digital displayed S-shaped tension transducer (Shavit scales, Haifa, Israel) to which a pulley was connected. A transmission wire connected to an electric winch (maximum power: 5 kg, 5 rounds per minute) was passed through the pulley, through the loops connected to the plaques' eyelets and back to form a knot on itself. The system was designed so that the weakest point was set to be the connection between the plaque and the eye. After turning the electric winch on, the wire stretched, the plaque eventually detached from the eye and the maximal power value was recorded on the digital display.

The weights needed to detach the plaques were 0.032±0.004 kg (range: 0.026-0.035 kg), 0.349±0.173 kg (range: 0.192-0.624 kg) and 0.405±0.083 kg (range: 0.298-0.498 kg) for the eyes treated with glue without conjunctiva, the eyes treated with glue with conjunctiva and the eyes treated with sutures, respectively. Using glue with no conjunctiva that covers the plaque resulted in significantly weaker adhesion strength compared to the 2 other groups (p value≦0.015), whereas no statistical differences were found between the glue with conjunctiva and sutures (p value=0.590).

### Example 2

### Plasminogen Activator Facilitates Detachment of a Plaque Affixed to a Sclera of an Eye through Fibrinogen Glue

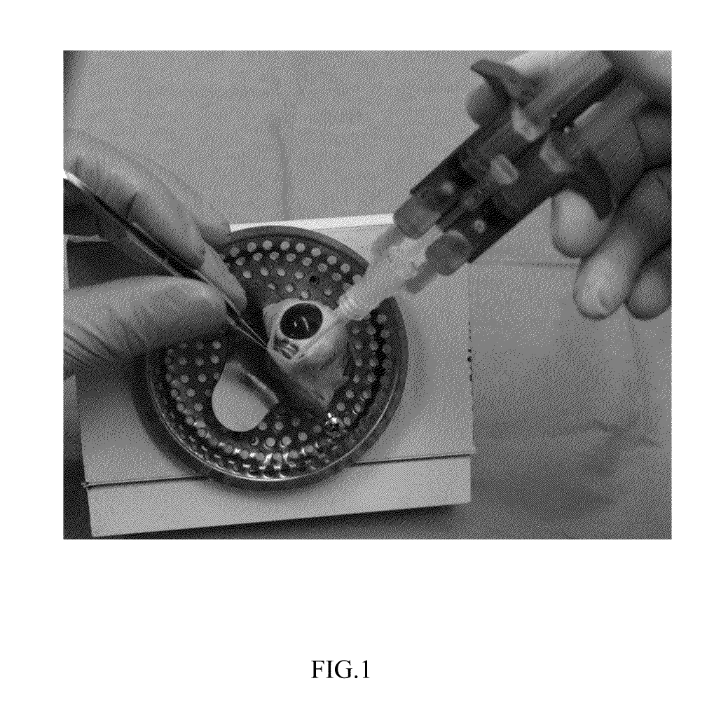

In order to examine whether a plasminogen activator is able to facilitate detachment of a plaque affixed to a sclera through fibrinogen glue, six extracted porcine eyes were used. The eyes were prepared as described in Example 1, followed by affixing 12 mm COMS-style gold plaques (Eckert & Ziegler BEBIG, Berlin, Germany) to the eyes as described for the group treated with fibrin glue and conjunctiva in Example 1. Administration of the fibrin glue on the gold plaques is demonstrated in FIG. 1. The eyes with the affixed plaques were then incubated in plasma (MDAIS, Ramat Gan, Israel) for 3 days (72 hours) at room temperature to simulate a fibrinolytic environment (the plasma was replaced every 36 hours).

In order to examine whether a composition containing a plasminogen activator is able to detach the affixed plaques, three eyes were treated with 10 ml of Normal Saline (NS) while the remaining eyes were treated with 10 ml NS +10,000 units (0.38±0.08 ml) of Urokinase.

The NS had no effect on the plaque-glue-eye complex, whereas the Urokinase dissolved the adhesion between the glue layer and surrounding tissues. The plaques were then easily extracted, after which a glue “blanket” was removed in full. No tissue defects were observed thereafter.

### Example 3

### Fibrin Glue is Able to Affix a Plaque to the Sclera of a Human Eye in-vitro

A section of a human sclera, derived from a donor, was placed in a petri dish and a 16 mm gold plaque (without radioactive seeds) has been placed on the sclera. Next, 2 ml of fibrin-glue components (Evicel, Johnson&Johnson) were dripped on the plaque such that the excess glue spilled on the sclera and petri dish. After approximately 1 minute, once the fibrin glue has set, the sclera-plaque-glue complex was covered in plasma to simulate a fibrinolytic environment, and incubated for three days as described in Example 2. The plaque remained strongly bound to the sclera after three days of incubation. Next, 10 ml of Urokinase solution, as described above, was applied on the hardened fibrin glue. The fibrin glue broke down within 2-3 minutes, enabling extraction of the plaque from the eye.

### Example 4

### Fibrin Glue is Able to Affix a Plaque to the Sclera of an Eye in-vivo

An in-vivo animal model experiment was carried out to test the feasibility of the novel surgical technique, including plaque placement with fibrin glue and removal with urokinase.

Methods:

All animal experiments were performed in accordance with the ARVO Statement for the Use of Animals in Ophthalmic and Visual Research and approved by the Animal Care and Use Committee at Sheba Medical Center, Israel. An in-vivo animal model was used in 3 experiments, and the study animals were New Zealand white rabbits (Harlan Laboratories Ltd, Jerusalem, Israel). Each animal was housed in a separate cage under conventional conditions and fed chow and water ad-libitum. Prior to undergoing the in-vivo experiments, the rabbits were anesthetized by intramuscular injection of a mixture of 0.5 mg/kg ketamine (100 mg/ml; Ketaset, Animal Health, Fort Dodge, Iowa, USA) and 10 mg/ml xylazine (20 mg/ml; Chanazine, Chanelle Pharmaceuticals Manufacturing, Galway, Ireland). The experimental eye was desensitized by a drop of oxybuprocaine (Dr. Fischer, Bnei Barak, Israel).

Surgical Technique Description:

The surgical technique included placing a 12 mm Collaborative Ocular Melanoma Study (COMS)-style gold plaque (Eckert & Ziegler BEBIG, Berlin, Germany) on the episclera after performing a 360° peritomy and after undermining beneath the Tenon capsule. A fibrin glue set (Tisseel, Baxter Corporation, Mississauga, Canada) was used as an adhesive component, and approximately 1 cc of glue was applied on top of and around the plaque, but not beneath it. The conjunctiva was then closed with 7/0 Vicryl sutures, and drops of Dexamycin (Neomycin—Dexamethasone, Teva Pharmaceutical Industries Ltd, Petach Tikva, Israel) and Oflox (Ofloxacin, Cipla, Mumbai, India) were administered.

Depending on the experiment, the plaques were left affixed to the eye wall for a variable amount of time until they were removed. For plaque removal, the conjunctival sutures were cut and approximately 1 cc urokinase (medacGmbh, Hamburg, Germany; 10,000 units dissolved in 10 cc saline) was applied to dissolve the biological glue. After detachment of the plaque from its surroundings, it was removed together with glue remnants.

Plaque Fixation, Horizontal Movement and Tilting and Removal:

Plaque movement along the sclera (horizontal movement) and away from it (tilt) was measured by B-scan ultrasonography. Plaques were affixed as described above and left for 7-10 days in 8 rabbit eyes. Two of the 8 plaques contained I¹²⁵ radioactive seeds that were designed to deliver a dose of 79.99 Gy in 216 hours to an imaginary tumor apex located 4 mm from its base and at a dose rate of 0.37 Gy/h. The plaques were positioned on the episclera, posterior to the limbus in proximity to the posterior pole and at various anatomical locations in relation to extraocular muscles (EOMs). Three plaques had direct contact with EOMs, one was positioned under the inferior rectus and the eyelets of two were positioned under the superior rectus. In another case, a plaque was positioned posterior to a detached lateral rectus insertion, which was secured by a suture. Glue was applied as needed on both the plaque and the EOM whenever an EOM was involved. The plaques never had any contact with optic nerve sheaths. Because these eyes had no intraocular tumors, as a point of reference for ultrasonography measurements, prior to plaque insertion, a 20 gauge (G) scleral plug was inserted through the sclera and retinal layers after performing a sclerotomy with a 21G needle. Using a caliper that was dipped in Trypan blue, a 3-4 mm arc was stained on the episclera 6 mm anterior to the plug in order to mark the location of the plaque's anterior edge. The plaque was then placed on top of the plug, i.e., on the episclera, so that its location was approximately under the center of the plug. Longitudinal and transverse ultrasonographic sections were obtained at the time of plaque insertion. Distances from the plaque's edges (at its widest diameter) to the plug's sonographic signal in both transverse and longitudinal sections were measured to assess the plaque's horizontal movement. Longitudinal sections were also used to assess the posterior plaque tilt by measuring the distance between the outer scleral surface and the inner edge of the plaque at the level of the plaque's posterior edge. Ultrasonographic measurements were obtained again at the time of plaque removal, and differences in its location compared to that at insertion were analyzed. Removal of plaques was carried out as mentioned above. The rabbits were monitored for an additional 2 weeks for any signs of adverse reactions, with special attention to peri- and postoperative bleeding events.

Ocular Tissue Reaction to Fibrin Glue and Urokinase:

A total of 9 rabbit eyes were used for this in-vivo experiment in which the reaction of ocular surface tissues to fibrin glue and urokinase was tested and compared to normal saline (NS). One cc of fibrin glue was applied under the sub-Tenon capsule after gentle local dissection in 4 eyes. One cc of urokinase was applied in another 3 eyes, while 1 cc NS was applied to each of 2 additional eyes that served as controls. Plaques were not inserted in this experiment. Clinical follow-up for the examination of inflammation and/or bleeding events by a portable slit-lamp continued daily until animals were sacrificed. Three rabbits were sacrificed after substance application (day 0; 1 with fibrin glue, 1 with urokinase and 1 with NS), four rabbits after 7 days (1 with fibrin glue, 2 with urokinase and 1 with NS) and two rabbits after 21 days (2 with fibrin glue). A byproduct of this experiment was to follow the degradation process of fibrin glue in an extraocular milieu during 21 days of follow-up. After animals were sacrificed, the experimental eye was exenterated, underwent gross examinations, after which it was placed in 4% neutral buffered formaldehyde, and processed for light microscopic examination. Serial 5 μm thick sections were stained with hematoxylin and eosin (H&E) and examined by a specialist pathologist. Living animals also underwent magnetic resonance imaging (MRI) using a 1.5 Tesla machine at days 0, 7 and 21. Rabbits were positioned inside an MRI knee coil and underwent T1- and T2-weighted orbital scans, including fat suppression and 0.1 mmol\kg intravenous gadolinium injection (Bracco, Monroe Township, N.J., USA). A masked neuroradiologist (GG) examined the MRI sections to identify the sub-Tenon blebs, to look for the presence of any related inflammatory reactions, and to follow changes in the blebs over time. The urokinase-injected eyes were compared to the controls.

Impact of Fibrin Glue as an Orbital Space Occupier on Intraocular Pressure (IOP):

Two rabbit eyes were used in this in-vivo experiment and two independent and masked investigators (OZ and IDF) carried out 3 consecutive IOP measurements each using a TonoPen XL (Reichert Technologies, Depew, N.Y., USA). Only values with a coefficient of variation (standard deviation divided by the mean) ≦5% were accepted. IOP measurements were performed on day 0 at 3 time points: (1) after anesthetizing the animals and prior to inserting the plaque, (2) after performing a 360° peritomy, dissection of the Tenon capsule and placement of the plaque, and (3) 5 minutes after 1 cc fibrin glue was applied beneath the Tenon capsule and above the plaque. The glued plaques were left in place and additional measurements were performed on days 4 and 7. All measurements were taken around the same time in the morning. Differences in IOP measurements between investigators and before and after application of the glue (AIOP) were calculated and compared.

Statistical Analysis:

Statistical analysis, including matched pairs analysis, Student's t-test, and analysis of variance (ANOVA), was performed using Microsoft Excel 2007 (Microsoft Corporation, Redmond, Wash., USA) and SPSS software version 17.0 (SPSS, Inc., Chicago, Ill., USA). A P value <0.05 was considered statistically significant, and all data are presented with ±1 standard deviation (SD).

Results:

Plaque fixation, horizontal movement and tilting and removal:

The plaques in all 8 eyes were easily affixed with the fibrin glue. The glue did not reach the space between the plaque and episclera in any of the cases. This was accomplished by gently pressing the plaque to the episclera so that the space was sealed. An excess of glue covered the corneo-limbal border in 3 cases, however, it was easily removed with Westcott scissors.

Ultrasonography revealed that the plaques' horizontal movement was 0.5±0.2 mm (0.2-0.6 mm), and that there had been no plaque tilting in any case. Horizontal movements were 0.2 and 0.5 mm in the cases that contained radioactive sources and 0.2, 0.3 and 0.4 mm in the cases in which plaques were in contact with EOMs.

The plaques were left affixed for an average of 8.5±1.3 days (7-10 days). They were removed with 1.1 cc (0.9-1.2 cc) of urokinase. Application of urokinase enabled removal of the fibrin glue until the plaque was released from its surroundings and could be extracted. Inspection of the ocular tissues and the involved EOMs after plaque removal revealed only minor local inflammatory response that resolved without intervention after 3-5 days. Spontaneous ocular movement was observed in all cases during follow-up, including the detached EOM case, in which the EOM was sutured back to its original insertion after the glue had dissolved and the plaque had been removed. No bleeding events occurred during the acute or postoperative follow-up in any of the cases.

Horizontal movement may be of less concern when using sutures, however, posterior plaque tilting is, since it is reported to occur in approximately 50% of cases and may be related to local treatment failure. It should be emphasized that the plaques in the present study were deliberately placed as posteriorly as possible (refraining from direct contact with optic nerve sheaths), because the distance of sutured plaques to the posterior pole was found to be a risk factor for tilting

Ocular Tissue Reaction to Fibrin Glue and Urokinase:

The application of fibrin caused only a local and circumscribed inflammatory response throughout the follow-up. It was not possible to distinguish between the urokinase and NS cases by means of MRI scans, clinical observation or analysis of histopathology sections. Blebs were absorbed in a matter of minutes, there were no bleeding events during the immediate or postoperative follow-up, and the inflammatory response was minimal, local and limited in time (approximately 2 days) in both cases. The MRI T1 and T2 scans of the fibrin cases showed a hypointense extraocular cavity that molded the globe on day 0 and continued to do so on days 7 and 21. The interface between the episclera and conjunctiva was enhanced on T1 sections, together with fat suppression and contrast, in a relatively limited and defined area. H&E sections of ocular surface tissues containing fibrin glue at day 7 demonstrated a hyalinic, amorphic substance (i.e., fibrin glue) surrounded by a well-defined chronic inflammation process, mainly plasma cells and lymphocytes. After 21 days, the bleb was clinically determined as being circumscribed and smaller than its size on days 0 and 7, and the eye was observed as being quiet. Histopathology sections of the ocular tissues that were taken after 21 days showed a local increase in inflammatory cells to amorphic matter ratio as a result of fibrin glue degradation.

The use of urokinase was an efficient way to dissolve the glue and its adhesions to surrounding tissues (i.e., Tenon capsule and episclera) and to eventually remove the plaque and the glue remnants. Its application did not result in bleeding events or any other adverse effects. Based on clinical examinations, imaging findings and the results of histopathological studies, it may be concluded that urokinase can be safely used to dissolve a biological adhesive for purposes of plaque removal.

Impact of Fibrin Glue as an Orbital Space Occupier on Intraocular Pressure (IOP):

Differences in IOP measurements between the 2 investigators were 0.2±0.3 (0.1-0.7, matched pairs; P=0.118), therefore the values were averaged. No statistical differences were found in the AIOP between eyes with plaques prior to glue application on day 0 compared to eyes with plaques and glue on days 0, 4 and 7 (ANOVA; P>0.291).

### Example 5

### Impact of Radiation on Fibrin Glue and its Adhesion Properties

The adhesion strength of a plaque glued to an eye wall and exposed to measured quantities of radioactivity doses was measured and compared to sutured plaques in an ex-vivo animal model. Four enucleated porcine eyes were used (2 to which plaques were glued and 2 to which they were sutured) in this experiment. Glued plaques were affixed to the eye wall using a fibrin glue kit, as described above (Surgical technique description section). Sutured plaques were affixed to the eye wall using 5/0 nylon sutures (through eyelets 1, 3 and 5), in a manner similar to that of conventional practice. In addition, 2 4/0 silk sutures were passed through 2 of the plaque eyelets (numbers 2 and 4) in both the glued and sutured plaques and they were tied to create 2 loops. These loops were later used to connect the fibrin glue-plaque-eye wall complex to an adhesion strength-measuring device. The radiation source was a BrachyVision version 11.0.47 system (Varian Medical System, Palo Alto, Calif., USA), which is a treatment-planning system containing an iridium source (10 Curie) that allows planning high dose rate (HDR) treatments in a short time. HDR tubes were inserted through the eye wall and they exited through its other side, just beneath the affixed plaque (sutured or glued). When the tubes were in place under the plaque cavity, iridium seeds were inserted into them via a remote control system and the eye-glue-plaque complex was radiated. For this experiment, the system was programmed so that each of the 4 eyes received a radiation dose of 100 Gy in 489.5 seconds (0.14 hours) to an imaginary tumor apex located 4 mm from its base (dose rate of 714.29 Gy/h). After completion, each eye was attached through the previously described loops to an adhesion strength-measuring device, consisting of a digitally displayed tension transducer (Shavit Scales, Haifa, Israel), a pulley, a transmission wire and a winch. Using the measuring device, the plaques were forcefully detached from the eye wall and the maximal adhesion power value was recorded on the digital display. Adhesion strength measurements were compared between the sutured and glued plaques.

Adhesion strength measurements for the 2 irradiated glued eyes were 0.40±0.03 kg (0.38 kg and 0.42 kg). Adhesion strength measurements for the 2 irradiated sutured eyes were 0.39±0.00 kg (0.39 kg and 0.39 kg; Student's t-test, P =0.936).

It can be concluded that radioactivity did not have a weakening effect on the biological glue since the adhesion strength results were similar to those of sutures.

### Example 6

### Fibrin Glue does not Significantly Attenuate Radiation

A non-vivo experiment was carried out in order to test whether the glue causes attenuation of radiation, which may be due to, e.g. absorption and/or shifting the plaque away from the tumor.

Three separate drops of fibrin glue, approximately 1 mm thick each, were applied on a 3 mm thickness Perspex board attached to an Oncology EDR-2 film (Extended Dose Range Ready-Pack Film, Carestream Health, Inc, Rochester, NY, USA) on its opposite side. Three 12 mm COMS-style plaques containing I¹²⁵ seeds (Eckert & Ziegler BEBIG, Berlin, Germany) were pressed onto the fibrin glue mass so that the edges of the plaques were in contact with the Perspex board. The plaques and radioactive sources were left for one hour while the film blackened in response to the absorbed radiation. The same plaques were then placed directly on the board, with no glue to separate it from the radioactive sources, and left for an additional hour. The films were then developed, and the differences in absorption between the two groups (with and without fibrin glue) were compared using OmniPro-I′mRT software version 1.2 (IBA Dosimetry GmbH, Schwarzenbruck, Germany).

Smaller amounts of radiation reached the film when fibrin glue separated it from the radioactive sources (195 vs 170 cGy, 135 vs 115 cGy and 206 vs 145 cGy for the cases without and with glue for each pair, respectively). The differences between the cases with and without glue, however, were not significant (35.33±22.37 cGy, range: 25.00-61.00 cGy; matched pairs, P=0.111).

### Example 7

### Affixing a Plaque having a Concentric Annular Rim using Fibrin Glue

In order to examine the effect of adding a concentric annular ring to a gold plaque on the efficiency of affixing the plaque to the sclera using fibrin glue, five rabbits are used. In each rabbit, a gold plaque devoid of radioactive seeds is placed in both eyes, one plaque having a concentric annular rim and the other devoid of such a rim. All plaques are affixed to the sclera using a fibrin glue. A week following the insertion of the plaques to the eyes of the rabbits, a composition comprising tissue plasminogen activator (tPA) is administered to the eyes treated with the fibrin-glue to remove the plaque. Various parameters, such as adhesion strength of plaques with/without a concentric annular ring, are measured during the experiment.

What we claim is:

1. A method for reversibly affixing an implant to a sclera of an eye of a subject afflicted with a medical condition in the eye, the method comprising:
placing an implant on said sclera; and
applying a composition comprising at least one fibrin-based tissue adhesive to at least one of said implant and said sclera, thereby affixing said implant to the sclera.

2. The method of claim 1, wherein said method further comprises applying a composition comprising at least one plasminogen activator to at least one of said implant and said sclera following a desired time-period, thereby detaching said implant from said sclera; and removing said implant from said eye.

3. The method of claim 1, wherein said applying a composition comprising at least one fibrin-based tissue adhesive comprises applying a composition comprising fibrinogen and thrombin.

4. The method of claim 1, wherein applying said composition comprising at least one fibrin-based tissue adhesive to at least one of said implant and said sclera is applying said composition on top of said implant.

5. The method of claim 4, wherein applying said composition on top of said implant is configured to affix said implant to the sclera and to the conjunctiva of said eye.

6. The method of claim 2, wherein said plasminogen activator is selected from the group consisting of: tissue plasminogen activator, urokinase and a combination thereof.

7. The method of claim 1, wherein said implant is a plaque.

8. The method of claim 1, wherein said implant comprises a pharmaceutical composition.

9. The method of claim 8, wherein said pharmaceutical composition comprises a therapeutic agent selected from the group consisting of: an antibiotic agent, an anti-inflammatory agent, a micronutrient, a cytotoxic agent, a chemotherapeutic agent, a cytokine, a growth factor and a combination thereof.

10. The method of claim 1, wherein said implant comprises a radioactive source.

11. The method of claim 7, wherein said plaque comprises a protruding concentric annular rim.

12. The method of claim 11, wherein said composition comprising at least one fibrin-based tissue adhesive is configured to be positioned between said annular rim and the sclera.

13. The method of claim 11, wherein said protruding concentric annular rim is integrally formed with said plaque.

14. The method of claim 2, wherein said desired time period is between about 1-7 days.

15. The method of claim 1, wherein affixing to the sclera comprises affixing to the area between the sclera and conjunctiva of said eye.

16. The method of clam 1, wherein said medical condition is selected from the group consisting of: an eye neoplasm, choroidal neovascularization, myopia and a combination thereof.

17. The method of claim 16, wherein said eye neoplasm is selected from the group consisting of: malignant melanoma, intraocular lymphoma, retinoblastoma, medulloepithelioma, intraocular metastasis, Congenital retinal pigment epithelial hypertrophy (CHRPE), hemangioma and a combination thereof.

18. The method of claim 17, wherein said eye neoplasm is uveal melanoma.

19. A kit comprising:
at least one composition comprising at least one fibrin-based tissue adhesive;
a composition comprising at least one plasminogen activator; and
an implant comprising radioactive source.

20. The kit of claim 19, wherein said implant comprises a pharmaceutical composition comprises a therapeutic agent selected from the group consisting of:
an antibiotic agent, an anti-inflammatory agent, a micronutrient, a cytotoxic agent, a chemotherapeutic agent, a cytokine, a growth factor and a combination thereof.
